(19) United States
(12) Patent Application Publication
(10) Pub. No.: US 2005/0283241 A1
(43) Pub. Date: Dec. 22, 2005

(54) Intervertebral prosthesis for the cervical spine

(75) Inventors: Arnold Keller, Kayhude (DE); Paul C. McAfee, Baltimore, MD (US)

Correspondence Address: Barry E. Bretschneider;Morrison & Foerster LLP, Suite 300, 1650 Tysons Boulevard, McLean, VA, 22102, US

(73) Assignee: Cervitech, Inc., Rockaway, NJ (US)

(21) Appl. No.: 10/869,044

(22) Filed: Jun. 17, 2004

(51) Int. Cl. A61F 2/44

(52) U.S. Cl. 623/17.15

(57) ABSTRACT

A cervical vertebral prothesis includes a central part for bridging a central vertebral body and two outer prothesis parts which are engaged above and below the central vertebral body and are configured to be connected to vertebral bodies located above and below the central veterbral body. In order to secure both stability and mobility of the articulation of the assembled prosthesis, the central vertebral body is fixed to one of the outer prosthesis part and connected via a hinge to the other of the outer prothesis parts.

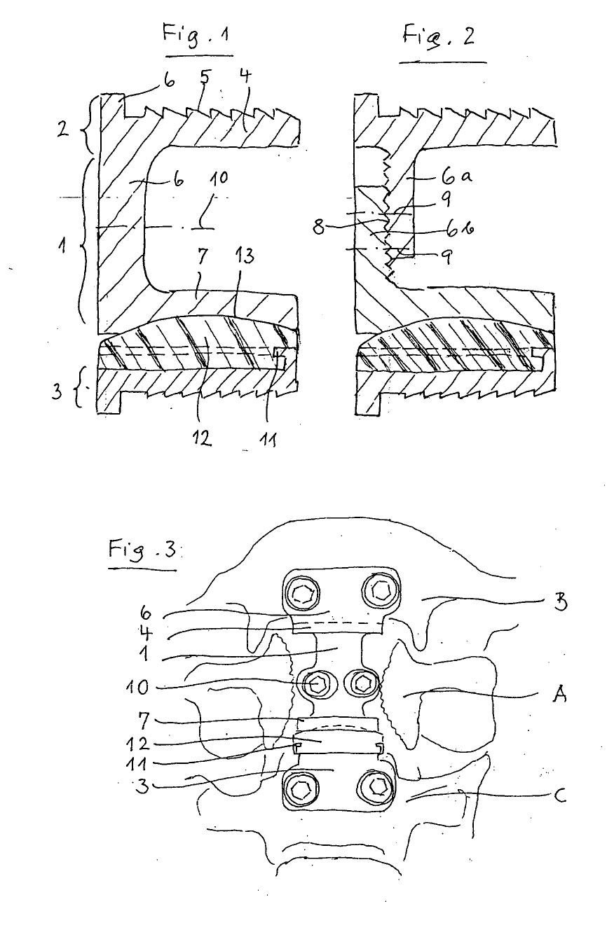

For replacement of a damaged intervertebral disk, endoprostheses are known which form a hinge (U.S. Pat. No. 5,534,029, FIGS. 1 and 2). It is also known to connect adjacent vertebrae fixedly to one another in order to eliminate a damaged intervertebral disk. This rigid connection can be promoted by means of so-called cages (U.S. Pat. No. 6,428,541, U.S. Pat. No. 5,772,661, U.S. Pat. No. 6,447,512 and other literature references cited therein). If two successive intervertebral disks and/or the vertebral body lying between them are damaged, the latter can be bridged with a two-stage prosthesis which includes two hinges for replacing the two intervertebral disks involved (EP-A-1417940, U.S. Pat. No. 5,534,029, FIG. 5; DE-A-4109941; DE-U-20115281 etc.). This has the disadvantage that the prosthesis tends toward a Z-shaped deformation if the two hinges buckle in opposite directions, or that the associated ligaments and articular facets are strongly stressed, which causes discomfort. This disadvantage is not found in two-stage prostheses in which both intervertebral disks involved are replaced or bridged (WO 9965412, WO 9201428). However, the double rigid connection of the vertebrae restricts the possibility of movement of the cervical spine.

The invention is intended to avoid this disadvantage and achieves this by the features of claim 1.

Accordingly, a prosthesis which bridges a central vertebral body and has connections to the two vertebral bodies adjacent to this central vertebral body is characterized in that one of these connections is fixed and the other is hinged. By virtue of the fixed connection of the central vertebral body to one of the two adjacent vertebral bodies, the prosthesis gives the cervical spine a high degree of stability. By virtue of the hinged connection of the central vertebral body to the other of the two adjacent vertebral bodies, the spinal column remains considerably more mobile than when using one of the known prostheses fixed on both sides.

To realize this design concept, the prosthesis expediently consists of a central prosthesis part to be connected to the central vertebral body or to replace the latter, and of two outer prosthesis parts to be connected to the adjacent vertebral bodies. The hinge is arranged between the central prosthesis part and one of the outer prosthesis parts. The other outer prosthesis part is connected fixedly to the central prosthesis part and can for example be designed in one piece with the latter.

The hinge expediently includes a sliding core which is connected fixedly to one of the adjacent prosthesis parts and interacts with the other one via a pair of sliding hinge surfaces.

For the connection of the outer prosthesis parts to the associated vertebral bodies, use can be made of various known constructions.

The invention is explained in more detail below with reference to the drawing, in which:

FIG. 1 shows a prosthesis in sagittal section;

FIG. 2 shows an embodiment with adjustable length, and

FIG. 3 shows an implanted prosthesis from the ventral direction.

The prosthesis consists of a central prosthesis part 1, an upper prosthesis part 2, and a lower prosthesis part 3. The central prosthesis part 1 is to be connected to the ventral face of a central vertebral body A or replaces the latter. The upper prosthesis part 2 is to be connected to a vertebral body B above the central vertebral body A, i.e. adjacent to the latter in the cranial direction. The lower prosthesis part 3 is to be connected to a vertebral body C located below the central vertebral body A, i.e. adjacent thereto in the caudal direction.

The upper prosthesis part 2 has a cover plate 4 whose top face 5 to be connected to the vertebral body is toothed in order to permit permanent connection with the bone tissue. Arranged at the ventral margin there is a flange or a ledge 6 which ensures that the end plate 4 adopts the position intended for it in relation to the vertebral body and cannot inadvertently shift in the dorsal direction. The flange 6 can have holes via which it is secured on the associated vertebral body by means of screws. The details can be similar to those of the prostheses disclosed in the publications EP 1344508 or WO 03/075804.

Generally speaking, the upper prosthesis part 2 is designed so that it can form a permanent and immovable connection with the associated vertebral body. It can therefore also have a form other than the one represented here, for example the form shown in the publication WO 92/01428, or with forms known from so-called cages (U.S. Pat. No. 6,447,545, U.S. Pat. No. 5,609,635, U.S. Pat. No. 6,224,595, U.S. Pat. No. 6,120,503, U.S. Pat. No. 6,123,705, U.S. Pat. No. 5,776,199).

The central part 1 consists of a bridge 6 and of a hinge plate 7. The bridge 6 is connected fixedly to the cover plate 4 and the hinge plate 7, namely in one piece in the example shown. In the embodiment according to FIG. 1, the bridge is made in one piece, so that the cover plate 4 and the hinge plate 7 are also integrally connected to one another. In the illustrative embodiment according to FIG. 2, the bridge 6 consists of two parts. Part 6a is integrally connected to the cover plate 4, and part 6b is integrally connected to the hinge plate 7. The parts 6a and 6b are designed in relation to one another in such a way that they can be connected fixedly and securely to one another in different length settings. In the example shown, they have a complementary toothing 8 consisting of parallel intermeshing transverse ribs. Screw connections are provided, indicated by broken lines 9, via which the bridge parts 6a and 6b are pressed against one another.

The divided design of the bridge 6 has the advantage that one prosthesis size can be used even with different spacings of the vertebral bodies between which they are intended to act. A further advantage is that the operating surgeon can fit the cover plate 4 without having to pay attention to the position of the hinge plate 7. This is because the bridge part 6b, with the prosthesis parts connected to it, can be placed onto the bridge part 6a from the ventral direction after the cover plate 4 with the bridge part 6a has been fitted.

The central part 1 is expediently connected fixedly and rigidly to the central vertebral body A if enough of the latter remains. For this purpose, at least one bone screw can be used, indicated by a dot-and-dash line 10. This possibility of securing it to the central vertebral body A can also be provided in the embodiment according to FIG. 2.

In terms of its connection to the associated vertebral body, the lower part 3 of the prosthesis can be like the upper prosthesis part 2. On the inside it forms, together with an undercut ledge 11, a securing means for a sliding core 12 made of a material that promotes sliding, for example polyethylene. The top face of the sliding core 12 and the bottom face of the hinge plate 7 form complementary, spherical slide surfaces 13 which allow the lower prosthesis part 3 a hinged movement in relation to the central and upper prosthesis parts 1, 2. Details of the hinge construction, in particular details of the connection of the sliding core 12 to the lower prosthesis part 3, can be taken in particular from published European patent application 1344508.

The sliding core 12 and/or the lower prosthesis part 3 can be made available in different heights in order to permit adaptation of the prosthesis height to different natural situations, even when the central prosthesis part 1 is made in one piece.

While FIGS. 1 and 2 are more diagrammatic, FIG. 3 shows details of the practical design in a view from the ventral direction. It is arranged on a central vertebral body A, a vertebral body B located above the latter, and a vertebral body C located below the central vertebral body A. The central part 1 of the prosthesis is firmly connected by screws 10 to the ventral face of the central vertebral body A, which is worked in order to permit better fitting. The cover plate 4 and the flange 6 forming the upper prosthesis part 2 are connected securely to the upper vertebral body B. Correspondingly, the lower prosthesis part 3 is connected securely to the lower vertebral body C. By means of the securing arrangements 11, it supports the sliding core 12 which forms a hinge with the hinge plate 7 of the central prosthesis part.

It may be stated in summary that the prosthesis connects the vertebral bodies B and C in an articulated manner by bridging the central vertebral body A. On the one hand, the vertebral column is thus given sufficient possibility of movement. On the other hand, the central vertebral body A is relieved of load and is secured in its position.

1. A cervical vertebral prosthesis comprising a central vertebral prosthesis part configured for bridging a central vertebral body and first and second outer prosthesis parts which are configured to be connected to vertebral bodies located, above and below the central vertebral body, wherein the central vertebral prosthesis part is fixed to the first outer prosthesis part and is connected to the second outer prosthesis part via a hinge.

2. The vertebral prosthesis as claimed in claim 1, wherein the first outer prosthesis part is made in one piece with the central prosthesis part.

3. The vertebral prosthesis as claimed in claim 1, wherein the hinge comprises a sliding core which is fixed to of either the first or second outer prosthesis part and interacts with the central vertebral prosthesis part via a pair of hinge surfaces.

4. The vertebral prosthesis as claimed in claim 1, wherein the central vertebral prosthesis part can be adjusted in length.

5. The vertebral prosthesis as claimed in claim 3, wherein the sliding core is available in different heights.
